(19) United States
(12) Patent Application Publication
(10) Pub. No.: US 2005/0256529 A1
(43) Pub. Date: Nov. 17, 2005

(54) Clip applier cartridge with internal ratchet

(76) Inventors: Haruyasu Yawata, Huntington Beach, CA (US); Russell E Ahlberg, Rancho Santa Margarita, CA (US); Edward D. Dolendo, Mission Viejo, CA (US)

Correspondence Address: APPLIED MEDICAL RESOUCES CORPORATION, 22872 Avenida Empresa, Rancho Santa Margarita, CA, 92688, US

(21) Appl. No.: 10/518,436

(22) PCT Filed: Jul. 21, 2003
(86) PCT No.: PCT/US03/22752

§ 371 Date: Dec. 16, 2004

Related U.S. Application Data
(60) Provisional application No. 60/398,189, filed on Jul. 23, 2002.

(51) Int. Cl. A61B 17/10

(52) U.S. Cl. 606/143

(57) ABSTRACT

A clip applier for applying a surgical clip in a patient comprising a disposable cartridge and a reusable actuating mechanism. The cartridge includes an elongate tube having a proximal end and a distal end, a pair of opposing jaw members extending outwardly from the distal end of the elongate tube, and a one-way ratchet mechanism providing a full actuating stroke. The actuating mechanism is coupled to the proximal end of the elongate tube to move the jaw members between an open position and a closed position. The ratchet mechanism may be formed from injection molded plastic. The cartridge further comprises a push member for advancing the clip into the jaw members, a biasing spring for maintaining the push member against the clip, and a drive coupling operatively connected to a sliding ratchet pawl for engaging with mating teeth formed on an inner surface of the elongate tube, wherein the ratchet pawl includes a cantilever arm that engages with the mating teeth, and wherein the drive coupling provides a central connection of the ratchet pawl allowing pivoting and improved seating of the mating teeth. A method of applying a surgical clip to an object in a patient using the clip applier of the invention includes the steps of positioning the clip applier within the patient such that the jaw members are adjacent to the object to be clipped, closing the jaw members and the clip over the object, and removing the clip applier from the patient. The method may further comprise the steps of orienting the clip applier such that the jaw members pass over the object, manipulating the jaw members to move the object to a compressed state within the jaw members, advancing the clip over the object to maintain the object in the compressed state, and retracting a feeder to allow the next clip to be fed into the jaw members.

### BACKGROUND OF THE INVENTION

1. Field of the Invention

This invention generally relates to surgical instruments and, more particularly, to clip appliers having a reusable portion and a disposable portion including a ratchet mechanism.

2. Discussion of the Prior Art

Clip appliers are frequently used in endoscopic surgery for the application of hemostatic clips. Several significant characteristics of such instruments include simplicity in construction, reliability in operation and low cost. The instruments need to be sufficiently economical to allow reusability and/or disposability. At the same time, the instruments need to provide surgeons with good tactile feedback and control during surgery.

Various surgical instruments have been developed which address some of the above needs. An example is a clip applier having a one-way ratchet mechanism where a user completes a full actuating stroke of the instrument before the engagement releases and returns to the original starting position. By incorporating the one-way ratchet mechanism in the applier and, more particularly, in the cartridge of the clip applier, the problems of clip drop and partial clip closure are significantly reduced. Until now, however, clip appliers have been either reusable or disposable with the one-way ratchet mechanism built into the handles. The main reason for placing the ratchet mechanism in the handle was because the ratchet mechanism comprises complicated mechanical components and there was simply more space in the handle. Drawbacks of the prior art design are it is complex, expensive to produce and difficult to clean/sterilize.

As such, there is a need in the art for a reliable, economical and yet compact clip applier having a reusable handle and a disposable cartridge wherein the ratchet mechanism is simplified and incorporated into the disposable cartridge to further reduce cost and improve reliability. An advantage of placing the one-way ratchet mechanism in the disposable cartridge is it simplifies the construction of the reusable handle. As a result, the new and improved clip applier handle contains fewer parts, is easier to clean/sterilize and is less expensive to produce. Another advantage of the clip applier handle is it may be used with a non-ratcheted cartridge.

### SUMMARY OF THE INVENTION

The invention is directed to a clip applier for applying a surgical clip in a patient comprising a disposable cartridge and a reusable actuating mechanism. The cartridge includes an elongate tube having a proximal end and a distal end, a pair of opposing jaw members extending outwardly from the distal end of the elongate tube, and a ratchet mechanism. The actuating mechanism is coupled to the proximal end of the elongate tube to move the jaw members between an open position and a closed position. The clip applier incorporates a one-way ratchet mechanism in the disposable cartridge that allows a user to complete a full actuating stroke before the engagement releases and returns to the original position. This results in reducing the negative effects of clip drop and partial clip closure.

The ratchet mechanism may be formed from injection molded plastic. The disposable cartridge further comprises a push member for advancing the clip into the jaw members, a biasing spring for maintaining the push member against the clip, and a drive coupling operatively connected to a sliding ratchet pawl for engaging with mating teeth formed on an inner surface of the elongate tube, wherein the ratchet pawl includes a cantilever arm that engages with the mating teeth, and wherein the drive coupling provides a central connection of the ratchet pawl allowing a degree of pivoting and improved seating of the mating teeth.

A method of applying a surgical clip to an object in a patient using the dip applier of the invention includes the steps of positioning the clip applier within the patient such that the jaw members are adjacent to the object to be clipped, closing the jaw members and the clip over the object, and removing the clip applier from the patient. Following the positioning step, the method may further comprise the steps of orienting the clip applier such that the jaw members pass over the object, manipulating the jaw members to move the object to a compressed state within the jaw members, and advancing the clip over the object to maintain the object in the compressed state. The method may further comprise the step of retracting a feeder to allow the next clip to be fed into the jaw members.

### DESCRIPTION OF THE DRAWINGS

FIG. 1 illustrates a reposable clip applier having a disposable cartridge and a reusable handle in accordance with a first embodiment of the invention;

FIG. 2 depicts an exploded view showing the assembly of the components of the disposable cartridge;

FIG. 3 illustrates the ratchet mechanism of the disposable cartridge in a fully assembled condition;

FIG. 4 depicts an exploded view showing the assembly of the ratchet mechanism of FIG. 3;

FIG. 5 illustrates the ratchet mechanism of FIG. 3 in a closed position;

FIG. 6 illustrates the ratchet mechanism of FIG. 3 in a returning position;

FIG. 7 illustrates the ratchet mechanism of FIG. 3 in an opened position;

FIG. 8 illustrates the ratchet mechanism of FIG. 3 in a closing position;

FIG. 9 illustrates a reposable vascular clip applier having a disposable cartridge and a reusable handle in accordance with another embodiment of the invention;

FIG. 10 depicts an exploded view showing the assembly of the components of the disposable cartridge of FIG. 9;

FIG. 11 illustrates the ratchet mechanism of the disposable cartridge in an assembled condition;

FIG. 12 illustrates the ratchet mechanism of FIG. 11 in an opened position;

FIG. 13 illustrates the ratchet mechanism of FIG. 11 in a returning position;

FIG. 14 illustrates the ratchet mechanism of FIG. 11 in a closed position; and

FIG. 15 illustrates the ratchet mechanism of FIG. 11 in a closing position.

### DESCRIPTION OF PREFERRED EMBODIMENTS AND BEST MODE OF THE INVENTION

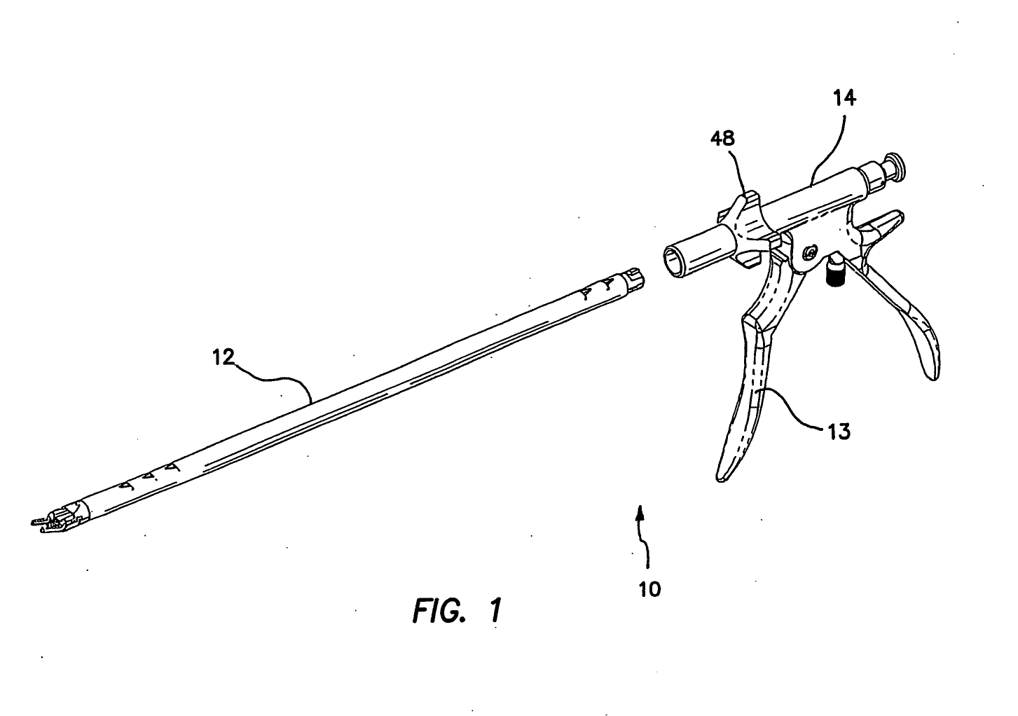

FIG. 1 illustrates a reposable laparoscopic clip applier 10 in accordance with the first embodiment of the invention. Reposable clip applier 10 is useful for endoscopic or less invasive surgery. Reposable clip applier 10 includes a disposable cartridge 12 and a reusable handle 14 directly applied to disposable cartridge 12. Reusable handle 14 includes a trigger 13 and a rotating knob 48 that can be used to reorient the position of disposable cartridge 12. A novelty of clip applier 10 is it incorporates a one-way ratchet mechanism in disposable cartridge 12 instead of reusable handle 14. An advantage of placing the one-way ratchet mechanism in disposable cartridge 12 is it simplifies the construction of reusable handle 14. As a result, clip applier 10 contains fewer parts, is easier to clean/sterilize and is less expensive to produce. Another advantage of clip applier 10 is since cartridge 12 is disposable, it does not need to be constructed to last as long as reusable handle 14. In operating clip applier 10, a user completes a full actuating stroke before the engagement releases and returns to the original position. This results in reducing the negative effects of clip drop and partial clip closure.

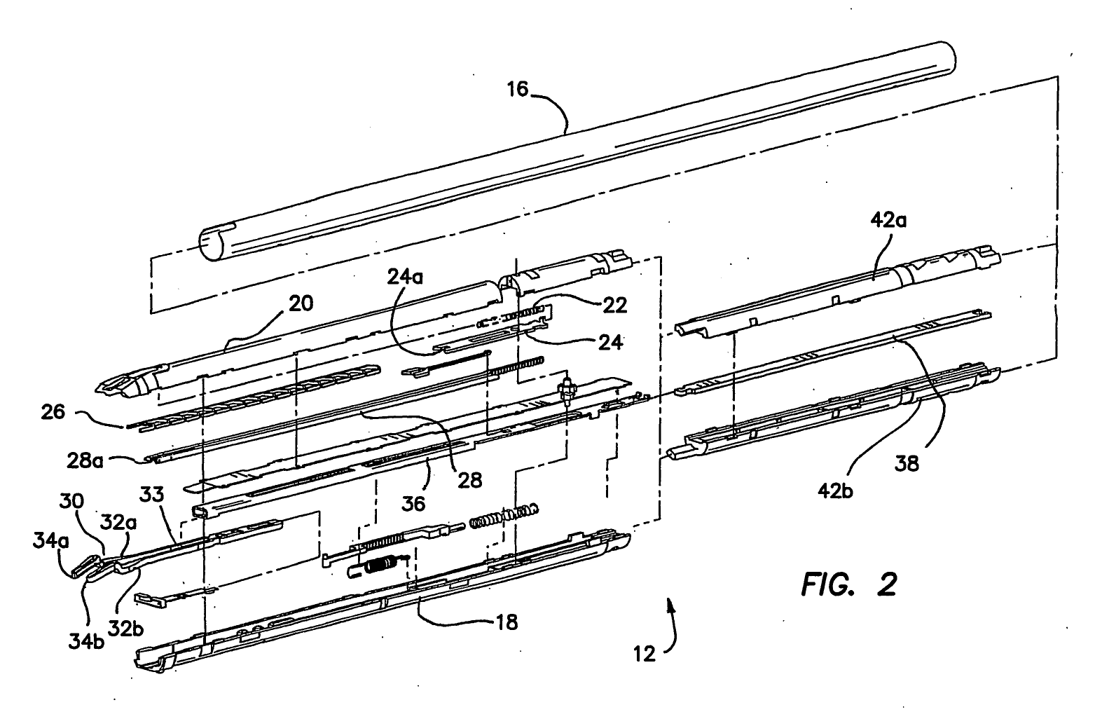

FIGS. 2-8 illustrate the details of the construction and operation of clip applier 10. Referring to FIG. 2, disposable cartridge 12 includes a cover tube 16 that holds a bottom housing 18 and a top cartridge 20 together. A pusher spring 22 is connected at its distal end to top cartridge 20 and at its proximal end to a pusher 24. The proximal end of pusher 24 has a pushing surface 24a that conforms to a clip 26. A series of clips 26 can be stacked end-to-end in front of pushing surface 24a; however, it is anticipated that the scope of the invention is broad enough to include a cartridge that applies one or more clips in sequence. Clips 26 rest on a feeder 28 as does pusher 24. The distal end of feeder 28 comprises a pushing surface 28a which, like pushing surface 24a, conforms to the shape of clips 26 for the purpose of further advancing the clips.

Disposable cartridge 12 further comprises a jaw 30, which has a pair of opposed tapered surfaces 32a and 32b at the distal end of an elongated slot 33. Mounted distally to tapered surfaces 32a and 32b are jaws or crimping members 34a and 34b, respectively. Closure member 36 is mounted within housings 18 and 20 and can translate responsive to a force input. Jaw 30 opens whenever handle 14 is relaxed enough to allow backward travel. While this arrangement is desirable for a skilled user demanding ultimate control of clip closure, it is more susceptible to clip drop and inadvertent partial clip closure. The present invention provides a one-way ratchet mechanism in disposable cartridge 12 directing a full actuating stroke of clip applier 10 so as to safeguard against clip drop and to assure complete closing of the clip.

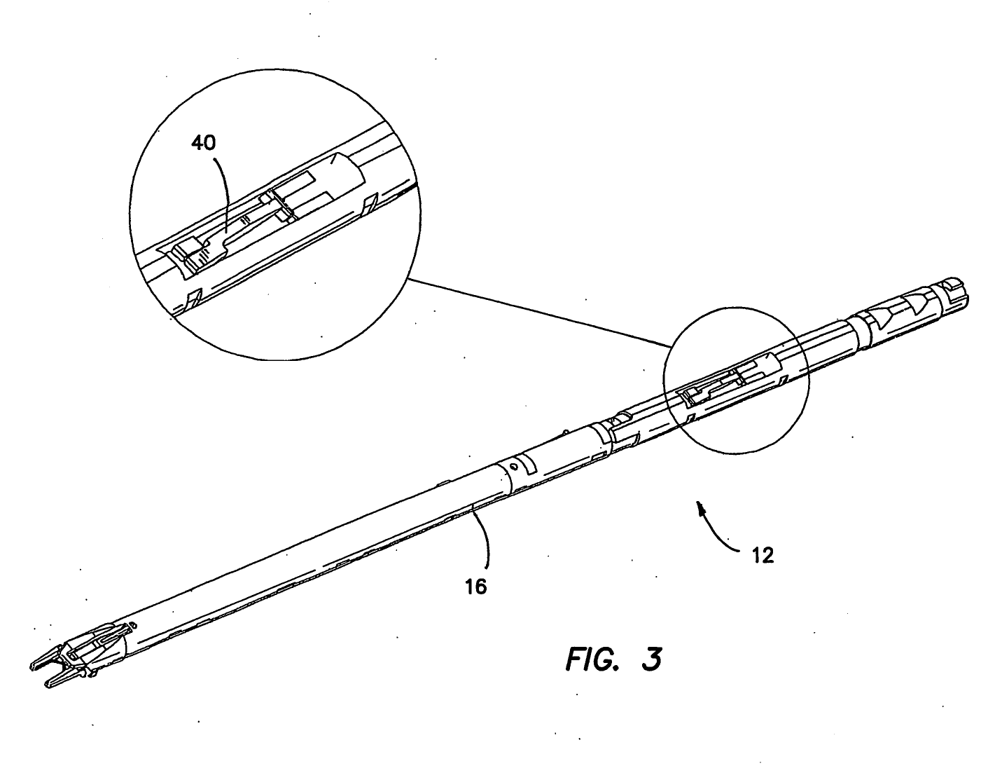

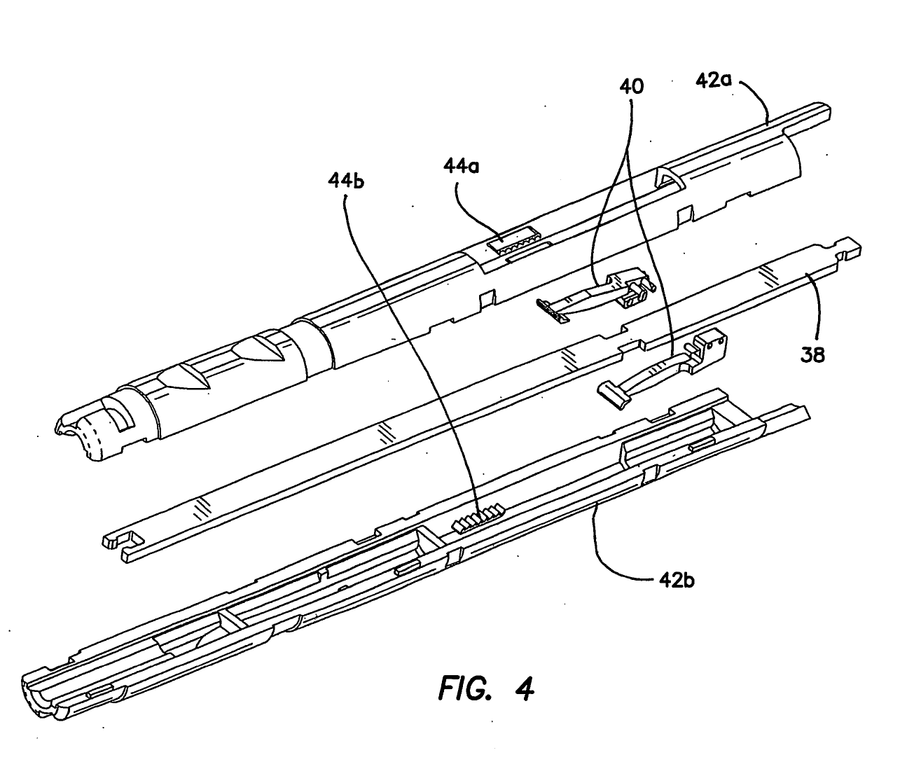

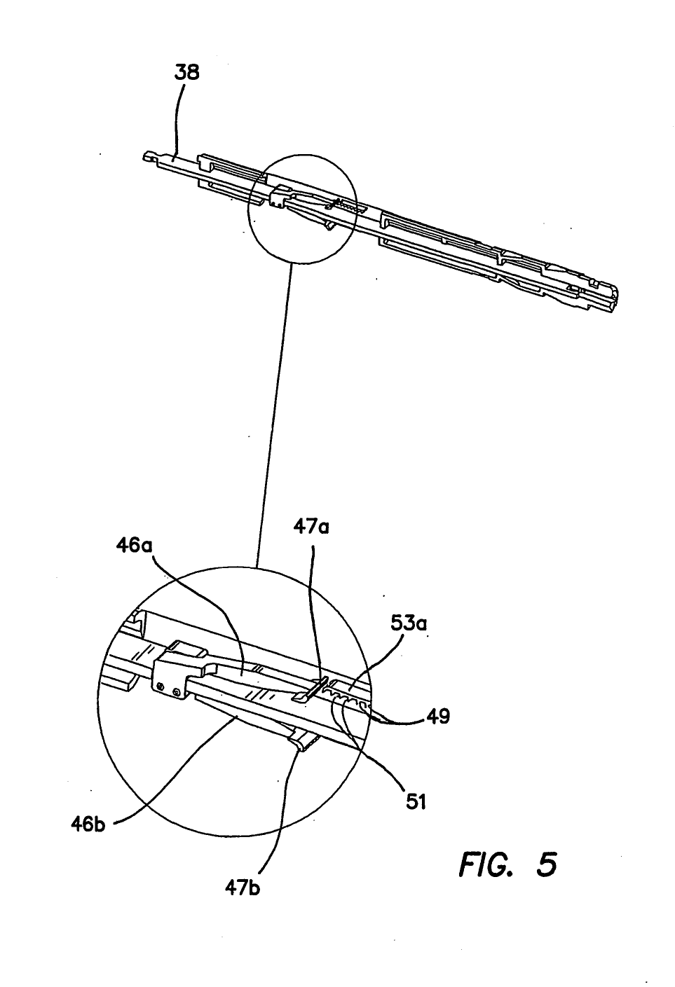

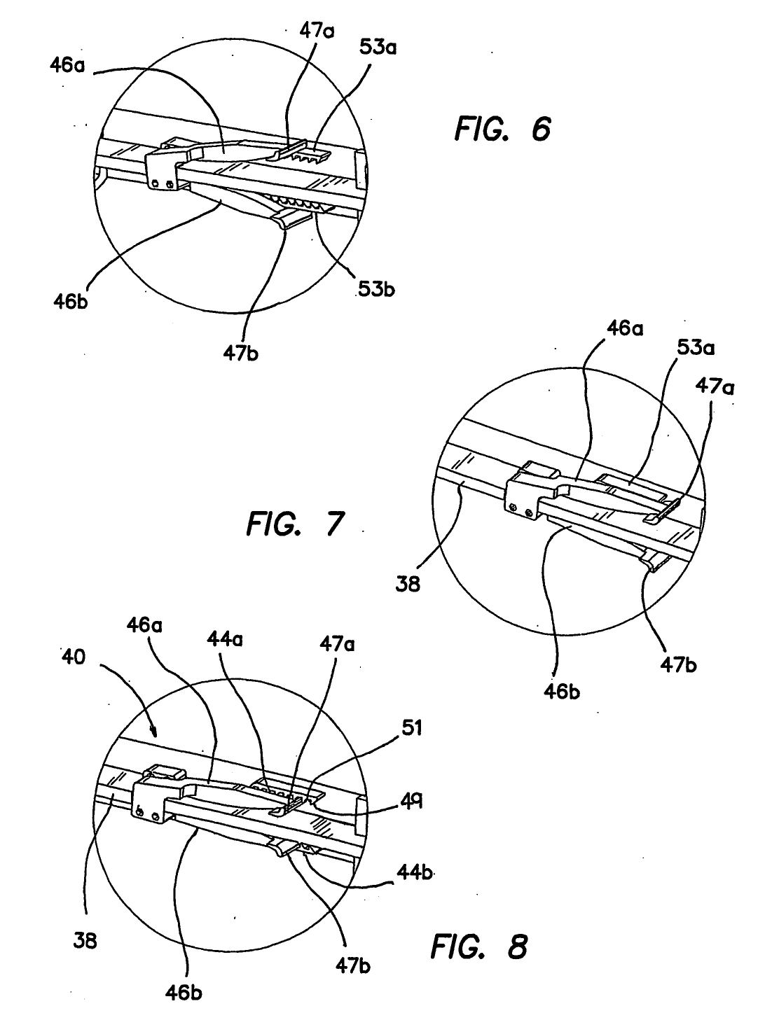

In particular, disposable cartridge 12 comprises a drive coupling 38 operatively connected to a sliding ratchet pawl 40 for engaging with ratchet teeth 44a and 44b of opposing inserts 42a and 42b, respectively, as illustrated in FIGS. 3 and 4. Ratchet pawl 40 includes a pair of cantilever arms 46a and 46b having sliding members 47a and 47b, respectively, that engage with ratchet teeth 44a and 44b, respectively, as further illustrated in FIGS. 5-8. Each tooth of ratchet teeth 44a and 44b comprises an inclined surface 49 followed by a straight side abutment 51. The inclined surface 49 allows a sliding member 47a/47b of a cantilever arm 46a/46b to slide in one direction but engages the side abutment 51 to prevent back travel. The number of tooth segments governs the length of the stroke. When ratchet pawl 40 reaches the end of the series of ratchet teeth 44a and 44b, sliding members 47a and 47b slide off the last tooth segments indicating completion of a full actuating stroke of clip applier 10 as illustrated in FIG. 5. Sliding members 47a and 47b then encircle back along surface planes 53a and 53b, respectively, as illustrated in FIG. 6 to return ratchet pawl 40 to its original opened position and starting point in FIG. 7.

In the above operation, cantilever arms 46a and 46b act as leaf springs to force sliding members 47a and 47b securely into ratchet teeth 44a and 44b, respectively. Each of cantilever arms 46a and 46b is operatively attached to drive coupling 38, which provides a central connection and allows a degree of pivoting to encourage better seating of the mating teeth. The central connection also allows for duplication of the elements on the reverse side of drive coupling 38, i.e., the second of the two cantilever arms can be engaged with the second set of the mirror-imaged teeth. An advantage of having a second set of ratchet teeth is it tends to equalize the forces on each side of the post as well as to dissipate the stresses by sharing the bearing forces. As a result, the ratchet design of the invention minimizes the tendency of cantilever arms 46a and 46b to torque. Moreover, reliability is also improved since the bearing forces are distributed evenly along the two sets of mirror-imaged components. That is, by having two sets of mirror-imaged components, the ratchet mechanism of the present invention cuts in half the loading on each individual component.

Ratchet pawl 40 and ratchet teeth 44a and 44b can be made with strong, compact and reliable components that are less bulky and lighter than metal components. For example, the design of the invention can be implemented by incorporating molded plastic parts. In one application of the invention, the ratchet mechanism is small enough to fit within the constraints of a 10 mm diameter cartridge barrel.

The operation of disposable cartridge 12 and process of positioning and feeding clips 26 will now be described. First, feeder 28 is distally extended so that a clip 26 is placed between crimping members 34a and 34b. Next, feeder 28 is retracted thus allowing pusher 24 to push the clip stack 26 forward moving the next clip in line to be fed and crimped. While feeder 28 is being retracted, closure member 36 advances toward tapered surfaces 32a and 32b. The force used to advance closure member 36 is supplied by pressing trigger 13 of handle 14. Clip 26, which was placed between crimping members or jaws 34a and 34b, is crimped as jaws 34a and 34b move toward each other when closure member 36 advances against tapered surfaces 32a and 32b pushing them together. Referring to FIGS. 5-8, pressing trigger 13 directs the full actuating stroke of the ratchet mechanism from a fully opened position in FIG. 7 to a closing position in FIG. 8, to a fully closed position in FIG. 5, to a returning position in FIG. 6, and then back to the fully opened position in FIG. 7.

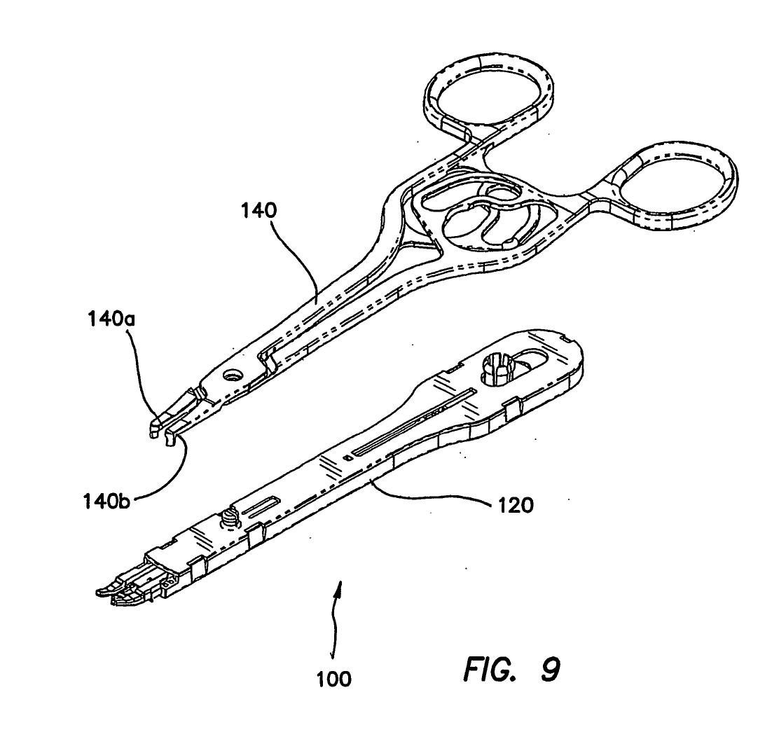

In another embodiment of the invention, FIG. 9 illustrates a reposable vascular clip applier 100 including a disposable cartridge 120 and a reusable handle 140 operatively connected to disposable cartridge 120. A novelty of clip applier 100 is it incorporates a one-way ratchet mechanism in disposable cartridge 120 instead of reusable handle 140. In a similar fashion to the laparoscopic clip applier, a user completes a full actuating stroke of clip applier 100 before the engagement mechanism releases and returns to the original position so as to reduce the negative effects of clip drop and partial clip closure.

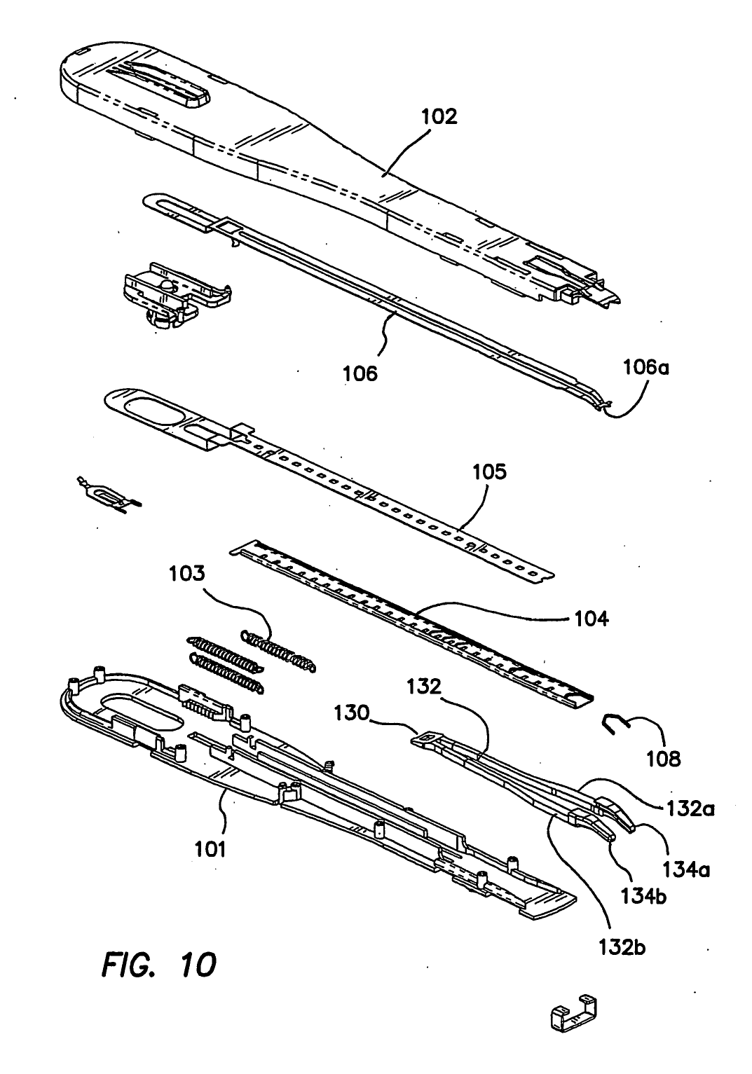

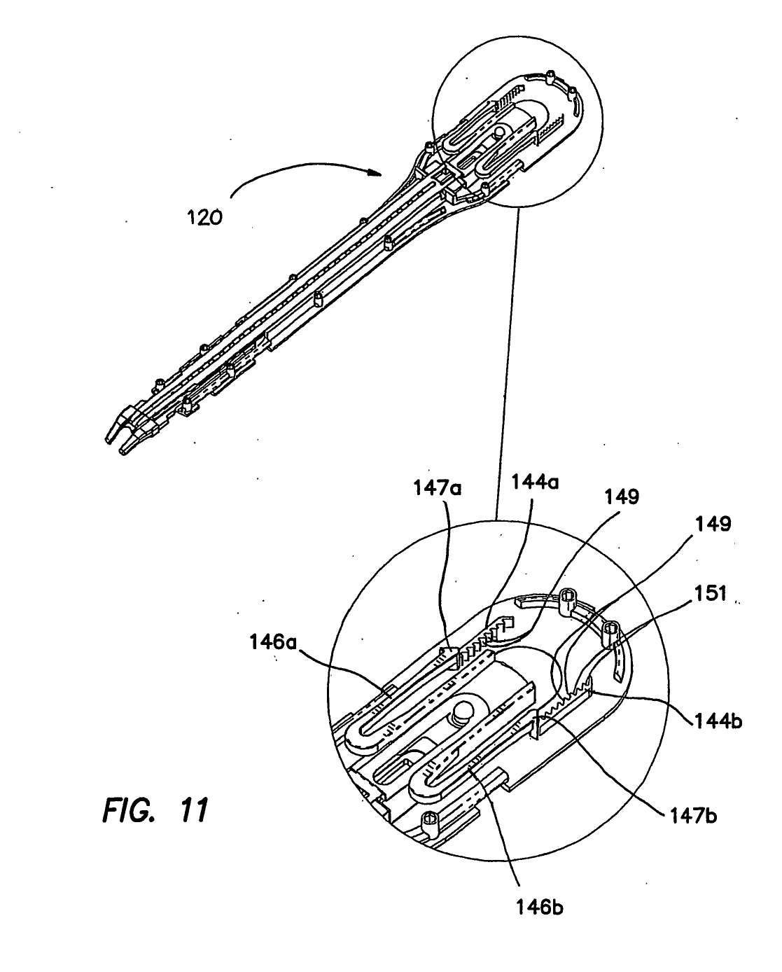

FIG. 10 depicts an exploded view of disposable cartridge 120 including a bottom housing 101 connected to a top housing 102. A spring extension 103 is connected at its distal end to bottom housing 101 and at its proximal end to a jaw loader 106. The distal end of jaw loader 106 has a pushing surface 106a that conforms to a clip 108. A series of clips 108 can be stacked end-to-end in a clip channel 104. An indexer 105 is provided to advance the next clip 108 in clip channel 104 into jaw loader 106. Disposable cartridge 120 further includes a jaw 130, which has a pair of opposed tapered surfaces 132a and 132b at the distal end of an elongated slot 132. Mounted distally to tapered surfaces 132a and 132b are jaws or crimping members 134a and 134b, respectively. Handle 140 includes tips 140a and 140b that open and close in response to movement of the handle. In particular, tips 140a and 140b close as handle 140 is squeezed and open after a full actuating stroke. The one-way ratchet mechanism of disposable cartridge 120 assures complete closing of each clip to safeguard against clip drop.

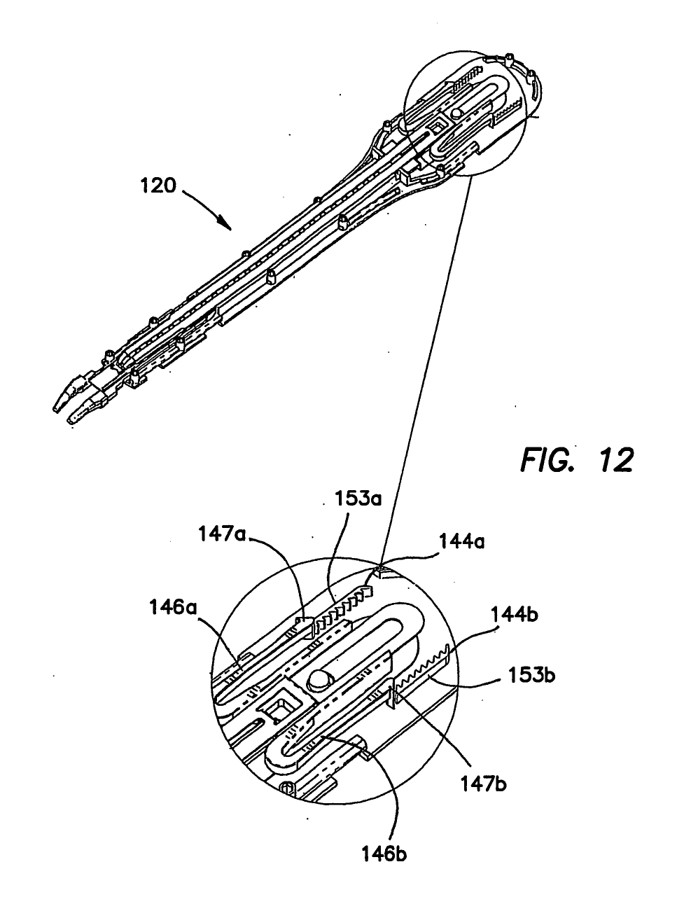

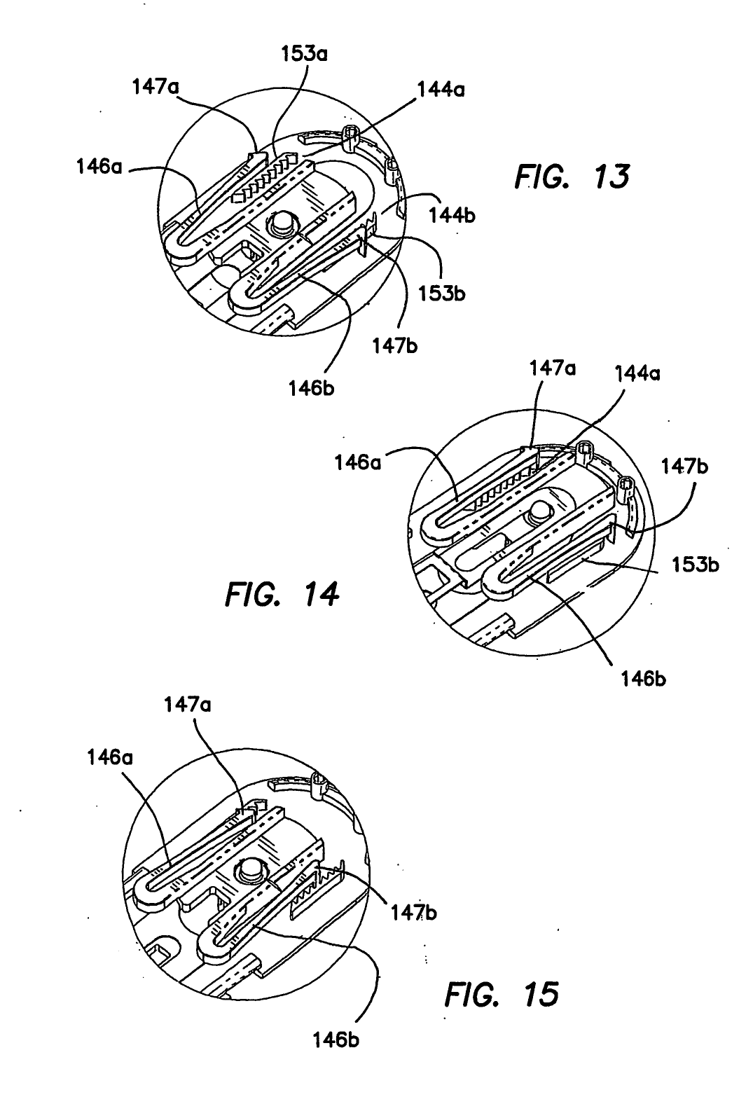

Disposable cartridge 120 further comprises a drive bushing 107 operatively connected to bottom housing 101, top housing 102, indexer 105 and jaw loader 106. Drive bushing 107 includes a pair of cantilever arms 146a and 146b having sliding members 147a and 147b, respectively, that engage and slide along mirror-imaged ratchet teeth 144a and 144b, respectively. Each tooth of ratchet teeth 144a and 144b comprises an inclined surface 149 followed by a straight side abutment 151. The inclined surface 149 allows a sliding member 147a/147b of a cantilever arm 146a/146b to slide in one direction but engages the side abutment 151 to prevent back travel. The number of tooth segments governs the length of the stroke. When sliding members 147a and 147b reach the end of the series of ratchet teeth 144a and 144b, respectively, sliding members 147a and 147b slide off the last tooth segments indicating completion of a full actuating stroke of clip applier 100 as illustrated in FIG. 14. Sliding members 147a and 147b then encircle back along surface planes 153a and 153b (non-teeth side), respectively, as illustrated in FIG. 13 to return cantilever arms 146a and 146b to their original opened position and starting point in FIG. 12.

In the above operation, cantilever arms 146a and 146b act as leaf springs to force sliding members 147a and 147b securely into ratchet teeth 144a and 144b, respectively. Cantilever arms 146a and 146b are mirror images of each other and are attached to drive bushing 107, which provides a central connection and allows a degree of pivoting to encourage better seating of the mating teeth. That is, the central connection allows for duplication of the,sliding members and ratchet teeth on opposite sides of drive bushing 107. An advantage of having two sliding members and corresponding ratchet teeth is it tends to equalize the forces on each side of the drive bushing as well as to dissipate the stresses by sharing the bearing forces. As a result, the ratchet design of the invention minimizes the tendency of cantilever arms 146a and 146b to torque. Moreover, reliability is also improved since the bearing forces are distributed evenly along the two sets of mirror-imaged components. By providing two sets of mirror-imaged components, the ratchet mechanism of the present invention cuts in half the loading on each individual component.

The operation of disposable cartridge 120 and process of positioning and feeding clips 108 will now be described. First, jaw loader 106 is distally extended so that a clip 108 is placed between crimping members 134a and 134b. Next, jaw loader 106 is retracted thus allowing indexer 105 to push the next clip in line to be fed and crimped. While jaw loader 106 is being retracted, handle tips 140a and 140b advance toward tapered surfaces 132a and 132b. The force used to close handle tips 140a and 140b is supplied by squeezing handle 140. Clip 108, which was placed between crimping members or jaws 134a and 134b, is crimped as jaws 134a and 134b move toward each other when handle tips 140a and 140b advance against tapered surfaces 132a and 132b pushing them together. Referring to FIGS. 12-15, squeezing handle 140 directs the full actuating stroke of the ratchet mechanism from a fully opened position in FIG. 12 to a closing position in FIG. 15, to a fully closed position in FIG. 14, to a returning position in FIG. 13, and then back to the fully opened position in FIG. 12.

As discussed above, a feature of the present invention is the ratchet pawl of the endoscopic clip applier and the drive bushing of the vascular clip applier can be made of injection molded plastic. In preferred embodiments of the invention, the plastic is stiff enough to counter the forces of the return spring to offset the property losses due to miniaturization. Moreover, the plastic is ductile enough to have spring-like properties. Gamma radiation resistance and lubricious characteristics would be an advantage. Viable resin candidates having the above characteristics include, among other materials, glass-filled nylon, liquid crystal polymers, and PEEK. Other plastics such as polyurethane, polyester, polycarbonate, polysulfone and polyetherimide in the high durometer range are also capable choices.

The foregoing disclosure and description of the invention are illustrative and explanatory thereof, and various changes in the size, shape and materials, as well as in the details of the illustrated construction, may be made without departing from the scope and spirit of the invention.

1. A clip applier for applying a surgical clip in a patient, comprising:
a disposable cartridge including an elongate tube having a proximal end and a distal end, a pair of opposing jaw members extending outwardly from the distal end of the elongate tube, and a ratchet mechanism; and
a reusable actuating mechanism coupled to the proximal end of the elongate tube to move the jaw members between an open position and a closed position.

2. The clip applier of claim 1, wherein the ratchet mechanism is a one-way ratchet providing a full actuating stroke of the clip applier.

3. The clip applier of claim 1, wherein the ratchet mechanism is formed from injection molded plastic.

4. The clip applier of claim 1, wherein said actuating mechanism comprises:
a main body connected to the proximal end of the elongate tube; and
an actuating handle coupled to the main body such that movement of the handle in relation to the main body forces the jaw members to move relative to each other between the open position and the closed position.

5. The clip applier of claim 1, wherein said cartridge further comprises a push member for advancing the clip into the jaw members.

6. The clip applier of claim 5, wherein said cartridge further comprises a biasing spring for maintaining said push member against the clip.

7. The clip applier of claim 6, wherein said cartridge further comprises a drive coupling operatively connected to a sliding ratchet pawl for engaging with mating teeth formed on an inner surface of the elongate tube.

8. The clip applier of claim 7, wherein the ratchet pawl includes a cantilever arm that engages with the mating teeth.

9. The clip applier of claim 7, wherein the drive coupling provides a central connection of the ratchet pawl allowing a degree of pivoting and improved seating of the mating teeth.

10. A method of applying a surgical clip to an object in a patient during a surgical procedure using a clip applier having a disposable cartridge and a reusable actuating mechanism, said disposable cartridge including an elongate tube having a proximal end and a distal end, a pair of opposing jaw members extending outwardly from the distal end of the elongate tube, and a ratchet mechanism, said actuating mechanism coupled to the proximal end of the elongate tube to move the jaw members between an open position and a closed position, said method comprising the steps of:
positioning the clip applier within the patient such that the jaw members are adjacent to the object to be clipped;
closing the jaw members and the clip over the object; and
removing the clip applier from the patient.

11. The method of claim 10, wherein the clip applier provides a full actuating stroke of the ratchet mechanism before returning to its original position by pressing the actuating mechanism.

12. The method of claim 10, wherein said actuating mechanism comprises:
a main body connected to the proximal end of the elongate tube; and
an actuating handle coupled to the main body such that movement of the, handle in relation to the main body forces the jaw members to move relative to each other between the open position and the closed position.

13. The method of claim 10, following the positioning step, further comprising the steps of:
orienting the clip applier such that the jaw members pass over the object;
manipulating the jaw members to move the object to a compressed state within the jaw members; and
advancing the clip over the object to maintain the object in the compressed state.

14. The method of claim 13, further comprising the step of retracting a feeder to allow the next clip to be fed into the jaw members.

15. A clip applier for applying a surgical clip in a patient, comprising:
a disposable cartridge including a housing member having a proximal portion and a distal portion, a pair of opposing jaw members extending outwardly from the distal portion of the housing member, and a ratchet mechanism; and
a reusable actuating mechanism coupled to the cartridge to move the jaw members between an open position and a closed position.

16. The clip applier of claim 15, wherein the ratchet mechanism is a one-way ratchet providing a full actuating stroke of the clip applier.

17. The clip applier of claim 15, wherein the ratchet mechanism is formed from injection molded plastic.

18. The clip applier of claim 15, wherein said actuating mechanism comprises:
a plurality of tips connected to said jaws members; and
an actuating handle coupled to the tips such that movement of the handle in relation to the tips forces the jaw members to move relative to each other between the open position and the closed position.

19. The clip applier of claim 15, wherein said cartridge further comprises a jaw loader for advancing the clip into the jaw members.

20. The clip applier of claim 19, wherein said cartridge further comprises a biasing spring for maintaining said jaw loader against the clip.

21. The clip applier of claim 20, wherein said cartridge further comprises a drive bushing operatively connected to the housing member and to the jaw loader for engaging with mating teeth formed on an inner surface of the housing member.

22. The clip applier of claim 21, wherein the drive bushing includes a cantilever arm that engages with the mating teeth.

23. The clip applier of claim 21, wherein the drive bushing provides a central connection of the jaw loader allowing a degree of pivoting and improved seating of the mating teeth.

24. A method of applying a surgical clip to an object in a patient during a surgical procedure using a clip applier having a disposable cartridge and a reusable actuating mechanism, said disposable cartridge including a housing member having a proximal portion and a distal portion, a pair of opposing jaw members extending outwardly from the distal portion of the housing member, and a ratchet mechanism, said actuating mechanism coupled to the cartridge to move the jaw members between an open position and a closed position, said method comprising the steps of:
positioning the clip applier within the patient such that the jaw members are adjacent to the object to be clipped;
closing the jaw members and the clip over the object; and
removing the clip applier from the patient.

25. The method of claim 24, wherein the clip applier provides a full actuating stroke of the ratchet mechanism before returning to its original position by pressing the actuating mechanism.

26. The method of claim 24, following the positioning step, further comprising the steps of:
orienting the clip applier such that the jaw members,pass over the object;
manipulating the jaw members to move the object to a compressed state within the jaw members; and
advancing the clip over the object to maintain the object in the compressed state.

27. The method of claim 26, further comprising the step of retracting a jaw loader to allow the next clip to be fed into the jaw members.
